(19) United States
(12) Patent Application Publication
(10) Pub. No.: US 2005/0207939 A1
(43) Pub. Date: Sep. 22, 2005

(54) Water-quality assessment system

(76) Inventors: Christopher Roussi, Kalamazoo, MI (US); Robert A. Shuchman, Ann Arbor, MI (US); Guy A. Meadows, Pinckney, MI (US)

Correspondence Address: John G. Posa;Gifford, Krass, Groh, Sprinkle,, Anderson & Citkowski, P.C., 280 N. Old Woodward Ave., Suite 400, Birmingham, MI, 48009-5394, US

(21) Appl. No.: 11/006,304

(22) Filed: Dec. 6, 2004

Related U.S. Application Data
(60) Provisional application No. 60/527,349, filed on Dec. 5, 2003.

(51) Int. Cl. G01N 33/00

(52) U.S. Cl. 422/68.1

(57) ABSTRACT

A simple, effective and inexpensive water-quality measurement system comprises a housing suitable for travel on or in a body of water; one or more sensors for measuring water properties as the housing travels; a memory for storing information relating to the measured water properties; and software for processing measured properties. This invention combines into a single, inexpensive, and comprehensive system the sensors, computer hardware, signal-processing, Geographic Information System data, and Decision Support Software. This integration yields a system of novel and unparalleled utility. The water properties measured by the system may include: water temperature, water conductivity, pH level, dissolved oxygen, turbidity, water depth, salinity, total dissolved solids (TDS), ORP, chlorides, nitrates, and phosphates. A geographic information system (GIS) based on a global positioning satellite (GPS) technology is operative to determine geographic location and other factors, and an internal or external processor is operative to generate a map coordinating one or more of the listed characteristics to one or more of the water properties. A barometric pressure sensor may also be provided. In the preferred embodiment, the housing is a floating buoy.

### REFERENCE TO RELATED APPLICATION

This application claims priority from U.S. Provisional Patent Application Ser. No. 60/527,349, filed Dec. 5, 2003, the entire content of which is incorporated herein by reference.

### FIELD OF THE INVENTION

The invention relates generally to water quality measurement apparatus and, in particular, to a free-floating, water quality measuring and watershed evaluation system.

### BACKGROUND OF THE INVENTION

Water quality is a vital concern to any population. Not only is current assessment essentialy, the tracking of trends in quality is equally important. Since its inception in 1879, the U.S. Geological Survey (USGS) has monitored and assessed the quality of streams and ground water in the United States. Today, USGS provides information on issues such as the suitability of water for public supply and irrigation, aquatic ecosystem health, effects of agriculture and urbanization on water resources, acid rain, and disposal of radioactive waste.

The National Water-Quality Assessment Program (NAWQA), which began in 1991, focuses on water quality in 42 major river basins and aquifer systems, referred to as “study units.” A study unit boundary frequently crosses state boundaries and usually encompasses more than 10,000 square kilometers (about 3,900 square miles). Collectively, the study units include water resources available to more than 60 percent of the population served by municipal supply and irrigated agriculture in watersheds that cover about half of the land areas of the conterminous United States. NAWQA was designed to analyze the condition of streams and ground water, including the factors affecting the condition and what changes in water quality are occurring over time. One-third of all study areas are intensively investigated at any given time for 3 to 4 years, and trends are assessed about every 10 years.

Another water quality assessment program is sponsored by CSREES, the Cooperative State Research, Education, and Extension Service. The CSREES National Water Quality Program, funded by the USDA CSREES National Program Office for Water Quality, is a member of the larger CSREES Integrated Research, Education, and Extension Competitive Grants Program. This program is often referred to as a “406” Program because of its legislative roots in Section 406 of the Agricultural Research, Extension, and Education Reform Act of 1998 (AREERA) (7 U.S.C. 7626), the legislation that authorized the Secretary of Agriculture to establish this competitive grants program. The roots of this program are found in the Cooperative Extension Water Quality Initiative that began circa 1989. A multitude of other assessment programs, including state-level programs, are also in place. The information gathered by these organizations provides a scientific basis for decision-making related to resource management and restoration, and evidence as to how people interact with their environment.

To assist with these and other initiatives, various testing apparatus has been developed, some of which float in water to obtain measurements. In the early 1970s, for example, a water quality profile measurement system capable of unattended operation was mounted on a buoy managed by the Scripps Institution of Oceanography, La Jolla, Calif. However, the efficiency and energy demands of the Scripps system were such that it was not capable of unattended operation over an extended period of time.

Various patents have also issued on the subject. U.S. Pat. No. 4,089,209 describes a remote water monitoring system that integrates the functions of sampling, sample preservation, sample analysis, data transmission and remote operation. The system employs a floating buoy carrying an antenna connected by lines to one or more sampling units containing several sample chambers. Receipt of a command signal actuates a solenoid to open an intake valve outward from the sampling unit and communicates the water sample to an identifiable sample chamber. Such response to each signal receipt is repeated until all sample chambers are filled in a sample unit. Each sample taken is analyzed by an electrochemical sensor for a specific property and the data obtained is transmitted to a remote sending and receiving station. Thereafter, the samples remain isolated in the sample chambers until the sampling unit is recovered and the samples removed for further laboratory analysis.

A system for obtaining a vertical profile of water quality in a body of water is disclosed in U.S. Pat. No. 4,157,657. The system includes a sensor for sensing predetermined characteristics of water and the depth of water at which the predetermined characteristics are sensed, a cable attached to the sensor for holding the sensor, and a winch assembly attached to the cable for deploying and retrieving the cable and the sensor in a body of water. A control circuit is coupled to the sensor and to the motor controller for causing the motor controller to brake and stop the motor in response to sensing a predetermined depth of water.

More recently, ORSANCO, the Ohio River Valley Water Sanitation Commission launched a new Ohio River data buoy called “AMI” (Advanced Measurement Initiative Buoy). ORSANCO received funding through the US EPA advanced monitoring initiative program to build a data buoy containing sensors that monitor real-time conditions in the Ohio River. A large steel hull was designed to house multiple instruments and telemetry equipment to transmit wireless data to a base computer. Spread spectrum radio technology ensures interference-free remote communication. The NexSens 4100 radio system allows data transmission up to 5 miles with reasonable line-of-sight.

The buoy is equipped with a heavy-duty antenna and two 20-W solar panels. The base station is equipped with an omni directional antenna, a base radio and a computer to collect data from the buoy. A multi-parameter water quality module is used to log temperature, conductivity, dissolved oxygen, pH, turbidity and chlorophyll data. The dissolved oxygen probe features a pulsed technology that eliminates the need for stirring. The turbidity sensor includes a self-cleaning wiper that allows long-term deployments with minimal maintenance requirements. For flow measurements, a dedicated instrument records current velocity directly below the buoy. To take physical water samples, the system is triggered by control software when water quality and flow parameters fall within a specified range.

Commercial products are also available, such as the Buoy Systems from Intermountain Environmental, Inc. The buoy houses a wireless datalogger/transmitter and a sealed rechargeable battery. The on-board measurement system measures water quality sensors and uses spread spectrum radio technology to transmit the data to a base station. The buoy can transmit data up to one mile with its standard 1/4 wave omni-directional antenna. Distances up to 10 miles can be reached using a higher gain 3 dB YAGI antenna. The on-board battery is recharged with a solar panel mounted on the buoy. Typical parameters monitored include Dissolved Oxygen, Conductivity/Salinity, Temperature, pH, ORP, Water Level and Turbidity. Constructed of polyethylene the buoy is capable of suspending up to 10 lbs. of instrumentation. The system is intended for water quality applications where unattended, long-term deployments are required in small reservoirs, ponds, or lagoons.

Despite the existence of systems of the type just described, the need remains for an inexpensive, free-floating, water quality measuring and watershed evaluation system.

### SUMMARY OF THE INVENTION

This invention improves upon existing designs by providing a simple, effective and inexpensive water-quality measurement system. The preferred embodiment comprises a housing suitable for travel on or in a body of water; one or more sensors for measuring water properties as the housing travels; a memory for storing information relating to the measured water properties; and software for processing measured properties. The processor may further be operative to condense multiple measured properties into a single number for more straightforward recording and/or transmission.

The water properties measured by the system may include one or more of the following:
water temperature,
water conductivity,
pH level,
dissolved oxygen,
turbidity,
water depth,
salinity,
total dissolved solids (TDS),
ORP,
chlorides,
nitrates, and
phosphates

The preferred embodiment further includes a geographic information system (GIS) based on a global positioning satellite (GPS) technology operative to determine one or more of the following:
geographic location (latitude and longitude),
speed,
quality metric,
number of satellites used,
time and date.

The system may further be equipped with an internal or external processor operative to generate a map coordinating one or more of the listed characteristics to one or more of the water properties. A barometric pressure sensor may also be provided.

In the preferred embodiment, the housing is a buoy, and the information relating to the measured water properties is stored at predetermined intervals. A user may select which sensor(s) to record at the time of deployment. With fewer sensors being logged, the maximum collection time increases. An optional wireless communication link allows for location of the housing and real-time readout of the measurement values.

System software may be used to perform one or more of the following functions:
specify which sensors to log,
select the sample rate,
calibrate all sensors, storing curves in NVRAM,
examine each sensor output,
begin a mission,
extract mission results, and
compute a single water-quality index (WQI) relating one or more of the measured parameters to a standard.

A decision support system (DSS) allows the user to easily interpret the measured data.

### BRIEF DESCRIPTION OF THE DRAWINGS

FIG. 1 is a drawing that shows a buoy structure according to the invention;

FIG. 2 is a schematic diagram of system electronics;

FIG. 3A is a plot of turbidity measured in the Kalamazoo River;

FIG. 3B plots the recorded conductivity; and

FIG. 3C shows the recorded D.O. in mg/L.

### DETAILED DESCRIPTION OF THE INVENTION

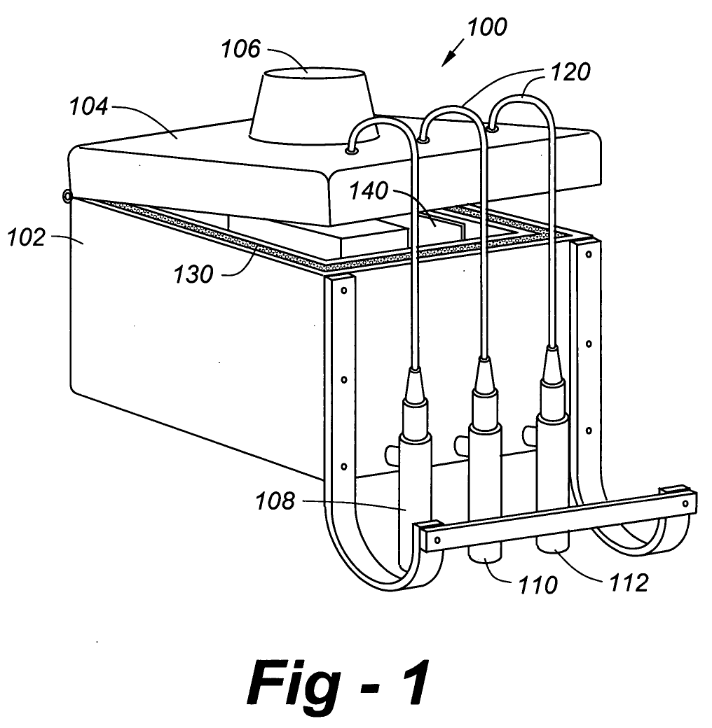

This invention is directed to an effective yet inexpensive water-quality assessment system, called the Automated Lagrangian Water-Quality Assessment System (ALWAS). Referring to FIG. 1, the apparatus includes a buoy 100, water-quality and water-parameter sensors 108, 110, 112, a microprocessor and recording device within the interior 140 of the buoy 100, geographic information system (GIS) interface software, and decision support system (DSS) software which generates water-quality maps based on the measurements.

The buoy 100 includes a roller portion 102 which is hinged to a lid 104. The lid has mounted thereon an antenna 106 associated with a global positioning satellite system described in further detail below, as well as sealed apertures 120 to carry signals from the sensors 108, 110, 112. An optional guard 122 is provided around the sensors to prevent debris from becoming entangled with the system.

The free-floating buoy may be equipped with more or fewer sensors than the three shown. The microprocessor measures and record environmental parameters needed for a complete surface-water characterization. The software processes the measured parameters into a form immediately useful in a GIS, as well as computing a water-quality index (WQI), which makes it convenient to interpret all the measurements as a single number. The software comprising the DSS is used to assist the user in making decisions based on the spatial and temporal parametric measurement of the surface-water quality.

Buoy Description

The buoy measures parameters at intervals determined by the user. GPS data input by the system may include geographic location (latitude and longitude), speed, quality metric, number of satellites used, time and date. Sampled water properties may include temperature, conductivity, pH, dissolved Oxygen, and turbidity. Other sensors may include sensors for depth, salinity, total dissolved solids, oxidation/reduction potential (ORP), chlorides, nitrates, phosphates, and bacterial count. Barometric pressure may also be monitored. A typical compliment of sensors is set forth in Table 1.

The buoy may be programmed to sample all on-board sensors at regular intervals, with the results being compressed and stored in on-board memory until retrieval. During collection, the location may be “spoken” via a text-to-speech module and relayed by standard cellular telephone to the user, to aid in retrieval of the buoy at the end of a mission.

Software on-board allows the user to specify which sensors to log; select the sample rate, calibrate all sensors, storing curves in NVRAM (non-volatile random-access memory); examine each sensor output; begin a mission; and dump mission values.

The prototype buoy measures approximately 25×20×20 cm, the size of a large lunch box, and costs two and five thousand dollars, depending on the sensor suite and on-board software capabilities. Turbidity is measured inside the buoy, with water pumped to the interior as needed.

TABLE 1
ALWAS Sensor Specifications

| Sensor | Value(s) | Units | Accuracy | Resolution |
|---|---|---|---|---|
| Garmin | time | UTM, hhmmss | N/A | N/A |
| GPS16A | | | | |
| | date | mmddyy | N/A | N/A |
| | latitude | ddmm.mmmm | <3 m | N/A |
| | longitude | dddmm.mmmm | <3 m | N/A |
| | Quality of | none | N/A | N/A |
| | reading | | | |
| | Number of | none | N/A | N/A |
| | satellites | | | |
| | speed | knots | <3 | 0.1 |
| DS1921L | temperature | ° C. | ±1° C. | 0.5 |
| iButton | | | | |
| VernierCON- | conductivity | 0-2000 μS/cm, or | ±1% FS | 1 |
| DIN | | 2 * mg/L TDS | | |
| Vernior DO- | Dissolved | 0-15 mg/L (ppm) | ±0.2 | 0.007 |
| DIN | Oxygen | | | |
| MPX4115A | Barometric | 0-115 kPa | 1.5% FS | 27.58 Pa |
| | pressure | | | |
| Vernier PH- | PH | 0-14 | 1.5% | 0.005 |
| DIN | | | | |
| Vernier TRB- | Turbidity | 0-200 NTU | ±2% <25 | 0.05 |
| DIN | | | NTU | |
| | | | ±5% >25 | |
| | | | NTU | |

Hardware Description

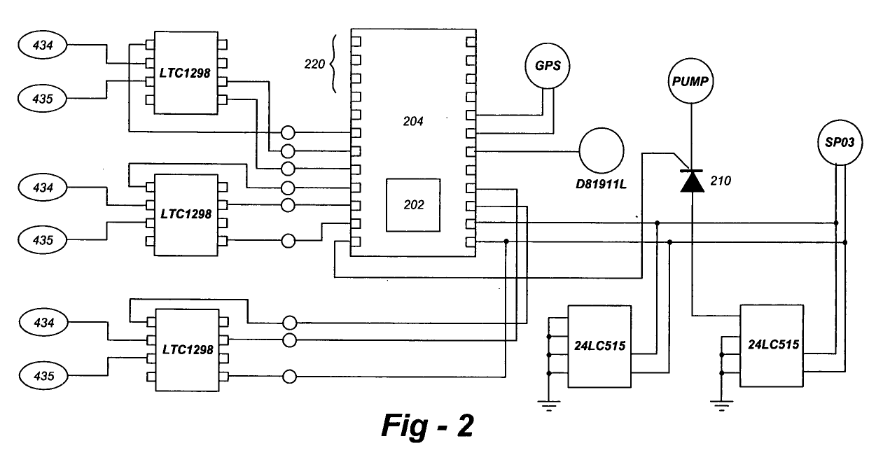

The electronics are based on a BS2pe single-chip microcontroller 202, as shown in the schematic of FIG. 2. Other integrated circuits mounted on module 204 include two 512K byte EEPROMs (electrically erasable programmable read-only memories) and several analog-to-digital (A/D) converters, including three 2-channel 12-bit A/D converters. The system further includes an integrated real-time clock and digital thermometer operated by a 10-year lithium battery (a “thermocron iButton” from Dallas Semiconductor). SCR 210 used to switch the water pump 212.

A serial port 220 is used to download the program which controls the processor, issue commands to set up a mission, upload the collected data, and to observe any debugging output from the running program. Sensor values are logged at a user-selectable rate into the on-board EEPROM. The storage capacity at this time is 1M bytes (enough for about 9 hours of collection with samples taken every 30 s).

Logged data are collected for further analysis through the serial port 220. Measurements of the completed circuit show that steady-state current draw is 305 mA nominally, with 470 mA being drawn when the pump is active. The pump runs for about 4 seconds in each 40 second collection interval, for ˜315 mA average current at 9 volts. With the current battery-pack rated for 6000 mAH, this implies a 19-hour maximum lifetime during collections.

Sensor Characteristics

The Global Positioning System is preferably a Garmin GPS16A, which is a self-contained, Wide Area Augmentation System (WAAS-enabled), GPS system for OEM use. It sends all of the standard NMEA sentences over an RS-232 interface. This is used to obtain the following measurements (which as logged in a packed format, to conserve storage):
UTM time (hhmmss)
Date (mmddyy)
Latitude (ddmm.mmmm) (we assume Western hemisphere)
Longitude (dddmm.mmmm) (we assume Northern hemisphere)
Valid/Invalid reading indicator (“A”=valid)
Quality metric
Number of satellites used in the computation
Speed (xxx.x in knots)

The unit can produce 1 complete measurement each second, although data may not be collected at that high a rate.

The digital thermometer used is a Maxim Thermocron iButton DS1921L, which is a self-contained digital thermometer with 0.5 deg. C. resolution, +/−1% accurate. It has a real-time clock, 9-year lithium battery, and is completely sealed. This sensor provides the temperature reading as needed.

The system further uses a Vernier Conductivity Probe (CON-DIN), which is a temperature-compensated, dip-type conductivity probe with epoxy body and parallel carbon (graphite) electrodes. This probe can be used to measure either water conductivity, or total ion concentration. The probe can be configured to these ranges:
Low: 0-200 μS/cm (0 to 100 mg/L TDS)
Mid: 0-2000 μS/cm (0 to 1000 mg/L TDS)
High: 0-20,000 μS/cm (0 to 10000 mg/L TDS)
   with these resolutions:
Low: 0.1 μS/cm
Mid: 1 μS/cm
High: 10 μS/cm

A Vernier Dissolved Oxygen Probe (DO-DIN) measures the concentration of dissolved oxygen. It is a Clark-type polarographic electrode, using a platinum cathode and a silver/silver chloride reference anode in KCl electrolyte. A gas-permeable plastic membrane separates the electrodes. The unit has these specifications:
Range: 0-15mg/L (ppm)
Accuracy: +/−0.2 mg/L
Resolution: 0.007 mg/L
Response time: 95% FS in 30 s, 98% FS in 45 s
Temperature compensated

The Motorola Integrated Pressure Sensor (MPX4115A/MPXA4115A series) piezoresistive transducer is a monolithic, signal conditioned, silicon pressure sensor.

This sensor combines advanced micromachining techniques, thin film metallization, and bipolar semiconductor processing to provide an accurate, high level analog output signal that is proportional to applied pressure. This will measure absolute pressure from 0 to 16.7 psi, with a maximum error of 1.5% FS. Using a 12-bit A/D, we achieve a resolution of 0.004 psi. The nominal transfer function is given by:
Vₒᵤₜ=Vₛ(0.009P −0.095)±(0.009PₑᵣᵣₒᵣT_{f}Vₛ
where Tf=1 over the operating range, and the pressure error factor is +/−1.5 kPa over the range of 15 kPa to 115 kPa (2.2 psi−16.7 psi).

For operation in the range of liquid water, the pressure is then given by:

$$P = \frac{{V}_{out} + 0.095 {V}_{s}}{0.009 {V}_{s}} = 111.11 \frac{{V}_{out}}{{V}_{s}} + 10.556$$

Using a 12-bit converter, with a regulated 5v supply, and including the specified error, this equation then becomes:
P=0.02713A_{d}+10.556±1.5(pressure in kPa)
where A_{d} is the A/D value.

A Vernier PH Probe (PH-DIN) 430 features a sealed, gel-filled, epoxy body Ag/AgCl sensor, capable of reading pH values from 0-14. The sensor voltage will be about 1.75v at pH=7, and will change by about 0.25v for each pH unit change. Response time is 90% of final reading in 1 s. The equation to convert from the voltage to pH is:
pH=−3.838V+13.720
or
pH=−0.004686A_{d}+13.720
depending on whether one reads voltage or the A/D output.

A Vernier Turbidity Sensor (TRB-DIN) (a nephelometer style) 432 measures the cloudiness of the water using an IR LED (890 nm). The amount of light that is scattered at right angles to the illumination is measured, and translated in turbidity units. According to the USGS, typical surface water readings would be from 0-50 NTU. The sensor can measure:
0-200 NTU
Accuracy
   +/−2 NTU for readings less than 25 NTU,
   +/−5% of readings above 25 NTU
12-bit resolution

A Text-to-Speech Module (Devantech SP03 text-to-speech synthsizer based on the WinBond WTS701EM/T chip) (not shown) is used to report the GPS position. Communication to the device is via the 12C protocol, supported by the microcontroller. During collections, each GPS collection is spoken aloud, in the format ddmm.mmmm (degrees and decimal minutes), as it comes out of the Garmin unit.

On-Board Software

System software is written in “PBASIC” for the Parallax Stamp chip. It is organized in several “slots” (2048 Byte memory pages) for addressing reasons. Briefly, as each sensor is called to produce a reading, the results are stored in ScratchPad RAM (which is common among all the running programs). These values are then packed into a more efficient storage scheme, written to non-volatile memory, and the process is repeated.

Slot 0—Main Program—This routine consists of the main control functions for calling each of the sensor programs, writing data to EEPROM, and performing the data retrieval at the end of a collection.

Slot 1—GPS and Temperature—This program reads several ASCII strings from the GPS, parses it to extract the information we desire, and puts these into SP RAM. The program then forces the ibutton to perform a temperature conversion, and reads that out. The value is converted to Celsius, and stored in SP RAM.

Slot 2—Conductivity—This program accesses the 0^{th} A/D chip, channel 0, and reads a 12-bit value that corresponds to the conductivity.

Slot 3—Calibration—This program is used to calibrate the conductivity and DO sensors 434, 436. The conductivity probe 434 is placed in a standard solution of 1000 mg/L NaCl, on the mid-range setting, and 100 values are averaged, the result being placed in SP RAM. The dissolved oxygen (DO) probe 436 needs both a low and high DO calibration, and these values are stored in SP RAM. When the program returns to the main program (slot 0), these values are transferred to EEPROM for more permanent storage (in case of power failure during a collection).

Slot 4—DO—This routine accesses the 1^{st} A/D chip, channel 0, to obtain the DO reading, which is stored in SP RAM.

Slot 5—Barometric pressure—This routine assumes that the pressure sensor is connected to the oth A/D, on channel 1.

Slot 6—Data dump routine—This routine outputs the collected data to the serial port in CSV format. Included in the output are the previously stored calibration data for this collection.

Slot 7—Turbidity and pH—This slot contains the code that collects the turbidity and pH. These were combined in this slot because they are individually simple routines, and this simplified the structure.

System Operation

When power is first applied, the hardware takes a reading from each sensor, and displays the results on the serial port, then waits for a user command. The main commands are:
0—perform a single collection and log the results at the current memory pointer
6—dump the data in memory
8—reset the starting memory address to 0000 and take a reading
12—calibrate all sensors
13—start a mission. This will continue until reset.

A computer with a serial port is needed to issue these commands at this time. During a typical collection cycle, the software takes the following steps:
1. GPS data are collected
   a. The NMEA sentence “GPRMC” is read and parsed to obtain most of the information needed.
   b. The NMEA sentence “GPGGA” is read to obtain quality values
   c. The latitude and longitude are reported audibly
2. the temperature is collected
3. conductivity is measured
4. D.O is measured
5. barometric pressure is measured
6. turbidity is measured
7. pH is measured
8. the reading in temporary memory are copied to EEPROM
9. addressed are updated for the next collection
   Processing and GIS Software

Once the data are collected and the mission has ended, the collected data are uploaded to a computer using the processing software. This performs these operations:
1. connects to the buoy microprocessor via the serial port and extracts all mission data
2. loads all data into a spreadsheet in an easy-to-read form
3. computes all calibration values, and loads a table with temperature and pressure compensated sensor values
4. computes a water-quality index based on selected sensor values
5. plots “quick-look” graphs of selected parameter sets
6. allows the user to examine, and edit, data values
7. writes selected sensor(s) in a form which the associated GIS software can ingest
   Decision Support System

This software extends the capabilities of the GIS system to allow users with specific questions the ability to make better use of the mission data. This is developed as needed, with specific knowledge used from the domains in which the decisions are being made.

### EXAMPLE

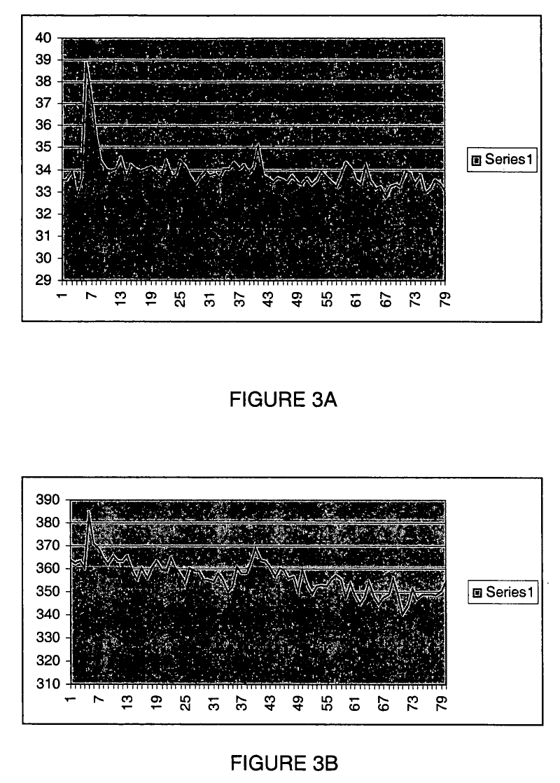

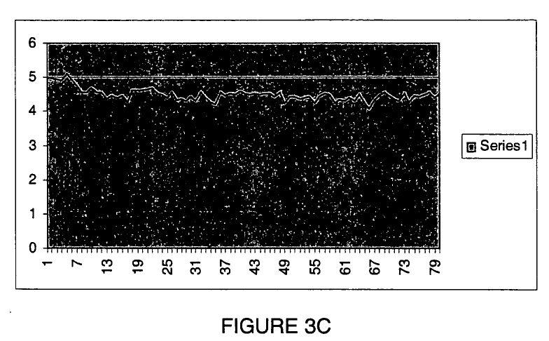

A collection was made on the Kalamazoo River, starting at N42.324, W85.36 for 60 minutes. FIG. 3A shows the measured turbidity. FIG. 3B plots the recorded conductivity, and FIG. 3C shows the recorded D.O. in mg/L.

1. A water-quality measurement system comprising:
a housing suitable for travel on or in a body of water;
one or more sensors for measuring water properties as the housing travels;
a memory for storing information relating to the measured water properties; and software for processing and condensing the multiple measured properties into a single number.

2. The water quality assessment system of claim 1, wherein the water properties include one or more of the following:
water temperature,
water conductivity,
pH level,
dissolved oxygen,
turbidity,
water depth,
salinity,
total dissolved solids (TDS),
ORP,
chlorides,
nitrates, and
phosphates.

3. The water quality assessment system of claim 1, further including a geographic information system (GIS).

4. The water quality assessment system of claim 1, wherein the GIS system is based on a global positioning satellite (GPS) technology.

5. The water quality assessment system of claim 4, wherein the GPS system is operative to determine one or more of the following:
geographic location (latitude and longitude),
speed,
quality metric,
number of satellites used, and
time and date.

6. The water quality assessment system of claim 5, further including an internal or external processor operative to generate a map coordinating one or more of the listed characteristics to one or more of the water properties.

7. The water quality assessment system of claim 1, further including a barometric pressure sensor.

8. The water quality assessment system of claim 1, wherein the housing is a buoy.

9. The water quality assessment system of claim 1, wherein the information relating to the measured water properties is stored at predetermined intervals.

10. The water quality assessment system of claim 1, wherein a user selects which sensors to record at the time of deployment.

11. The water quality assessment system of claim 1, wherein, with fewer sensors being logged, the maximum collection time increases.

12. The water quality assessment system of claim 1, further including the use of a wireless communication link to allow for location of the housing and real-time readout of the measurement values.

13. The water quality assessment system of claim 1, further including software to perform one or more of the following functions:
specify which sensors to log,
select the sample rate,
calibrate all sensors, storing curves in NVRAM,
examine each sensor output,
begin a mission, extract mission results, and
compute a single water-quality index (WQI) relating one or more of the measured
parameters to a standard.

14. The water quality assessment system of claim 1, further including software to perform the function of decision support (DSS), allowing to user to easily interpret the measured data.
